(19) United States
(12) Patent Application Publication
(10) Pub. No.: US 2007/0122301 A1
(43) Pub. Date: May 31, 2007

(54) Drywall mud pump

(76) Inventor: Werner Schlecht, Paradise Valley, AZ (US)

Correspondence Address: JENNINGS, STROUSS & SALMON, P.L.C., 201 E. WASHINGTON ST., 11TH FLOOR, PHOENIX, AZ, 85004, US

(21) Appl. No.: 11/292,238

(22) Filed: Nov. 30, 2005

(51) Int. Cl. A61M 1/00

(52) U.S. Cl. 417/437

(57) ABSTRACT

An improved capacity mud pump is achieved by lengthening the link between the piston and the pump handle in such a way that each stroke of the handle moves the piston farther, thus transferring more mud per stroke. Reconfiguration of the output aperture of the pump by attaching a high filler allows the user to hold the empty tool in a position that affords better leverage in working the pump handle and shaft guides improve the application of vertical force to the piston.

### FIELD OF THE INVENTION

This invention is in the field of small tools for the construction industry.

### BACKGROUND OF THE INVENTION

Drywall mud is a paste compound of calcium carbonate composites, such as mica, talc, and silica, that is used to fill corners, crevices, gaps and cracks in and between drywall sheets, often in conjunction with paper or fiberglass-mesh drywall tape. Drywall installers screw sheets in place and use mud and tape to form a smooth surface to be painted. Mud is applied in a paste or plastic state and dries in about 20 minutes if an accelerant is used, but otherwise can take several days, depending on the weather. Various specialized tools, such as tapers, flat boxes and angle boxes are used to apply the mud evenly and efficiently.

In practice, the drywaller uses pre-mixed mud or mixes a batch of mud in a container, such as a bucket, by adding water to the powdered material. He then transfers the mud to a hawk, or hand-held mud platform, and with a trowel applies the mud to the wall. Other tools, such as the automatic taper, the flat box and the angle box, include reservoirs for containing a quantity of mud and permit the drywaller to apply the mud directly to the wall. One common tool at construction sites is a mud pump, designed to fit over the edge of a bucket and capable of transferring mud into a tool with a mud reservoir for dispensing mud onto the wall.

A disadvantage of the existing mud pump is that its configuration forces the drywaller to stand bent over in an awkward position with one hand holding the tool being filled and the other moving the pump handle. It is an objective of the present invention to configure the pump so that the drywaller may be in a position to exert better leverage on the pump handle when filling a spreading tool. It is a further objective to increase the efficiency of the pump by transferring more mud per stroke of the handle.

### SUMMARY OF THE INVENTION

An improved capacity mud pump is achieved by lengthening the link between the piston and the pump handle in such a way that each stroke of the handle moves the piston farther, thus transferring more mud per stroke. Ergonomics are improved by raising the pivot point of the pump handle, lengthening the link to the handle and reconfiguring the output aperture by attaching a high filler that allows the user to hold the empty tool in a position that affords better leverage in working the pump handle. The wear characteristics and mechanical resistance to pumping are improved by adding a longer pump shaft guide inside the pump head.

### BRIEF DESCRIPTION OF THE DRAWINGS

FIG. 1 is a drawing of a prior art mud pump.

FIG. 2 is an exploded view of the prior art pump.

FIG. 3 is an exploded view of the improved pump of the present invention.

FIG. 4 is a multiple view of the pump with a high filler in place.

FIG. 5 is an illustration of the prior art pump in use filling a flat box.

FIG. 6 is an illustration of the present invention in use filling a flat box.

FIG. 7 is a detailed view of the high filler in FIG. 3.

### DETAILED DESCRIPTION OF THE INVENTION

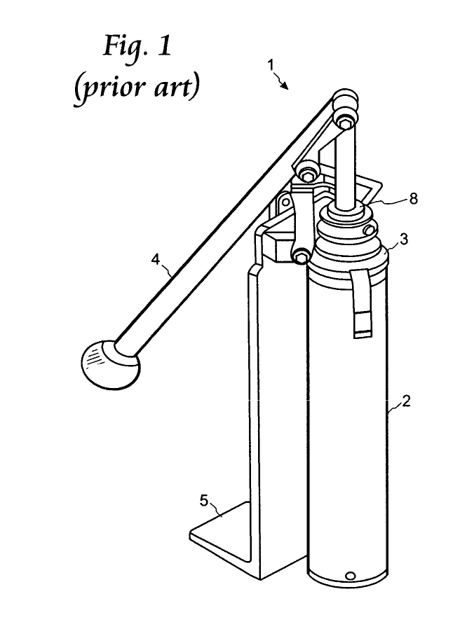

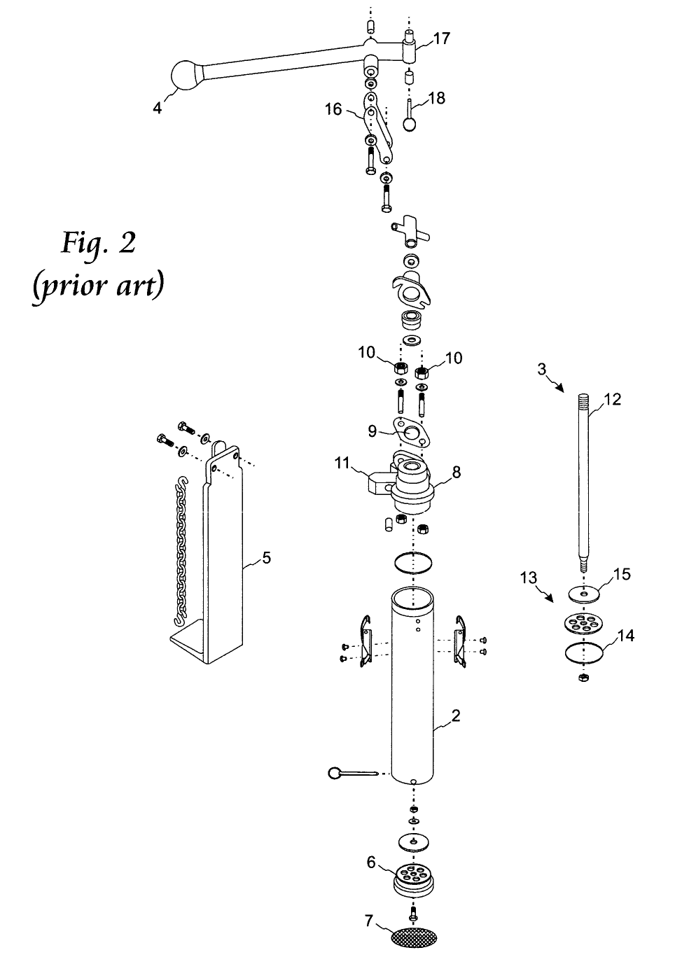

The configuration of a typical prior art pump is shown in FIGS. 1 and 2. Pump (1) comprises a main body tube (2), a piston (3) inside the main body tube, a pump head assembly (8), a handle (4) and a foot plate (5). In operation, the foot plate is placed outside the mud container and the main body tube is submerged in the mud.

Foot valve (6), protected by mesh screen (7), comprises the input through which mud enters the main body tube (2). Head assembly (8) includes an output aperture (9) with connection nuts (10) and an integral link base (11). Piston (3) includes piston rod (12) and piston head (13), made up of piston cup (14) and valve disc (15), that travel inside the main body tube (2).

Link member (16) connects the link base (11) on head assembly (8) to handle (4). Piston rod (12) passes through head assembly (8) and connects to the base (17) of handle (4) with a quick connect pin (18).

In operation, once the pump is primed, as the handle (4) is pushed downward, pulling piston rod (12) upward, mud is drawn into the main body tube (2) and mud above the piston cap (14) is expelled through output aperture (9), which is adapted to fittings designed to fill different tools. When handle (4) is pulled upward, pushing the piston rod (12) downward, valve (15) opens and allows mud to pass above the piston, to be expelled in the next stroke.

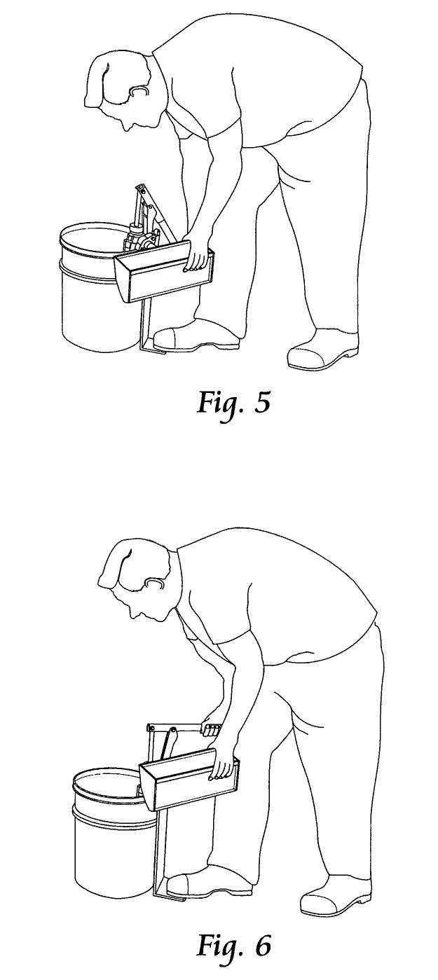

The prior art mud pump has certain disadvantages. When the drywaller holds a tool to be filled, such as a flat box, at the pump's output aperture, he must bend down to an awkwardly low position to mate the tool's input aperture to the output. Typically, when the handle is halfway through a stroke in a level position, the operator has to reach 16 inches toward the pump head and 4¼ inches down to hold the empty tool. As a result, the drywaller's other arm, which operates the pump handle, does not have an advantageous range of motion or very good leverage because his body is too close to the pump. See FIG. 5.

Another problem with prior art pumps is the stability of the pump shaft. Current pumps use a ¾ inch gland nut shaft guide in the top of the pump head. This allows the pump shaft to move horizontally in operation and puts a horizontal force on the shaft guide, causing wear on the pump cylinder, piston and shaft guide. It also requires application of more force on the handle to pump the mud.

The current invention improves the operation of a mud pump in three ways. First, the stroke of the piston is increased by increasing the length of link member (16) between the handle (4) and the link base (11). Average mud pumps known in the art are about 19½ inches high and have a piston stroke of about 4 inches. A lengthening of the link to 6 inches from the standard 4 inches causes the same piston to travel about 2 inches farther as the handle moves from its uppermost position to its lowermost.

Raising the link base by about 1 inch on the pump head, when combined with a 2 inch increase in the length of the link, has the effect of raising the pivot point of the pump handle by 3 inches. The higher location increases the difference in elevation between the operator's handle-gripping hand and his hand that holds in place the empty tool and the output aperture. This differential distorts the body position of the operator and decreases leverage and range of motion in working the handle.

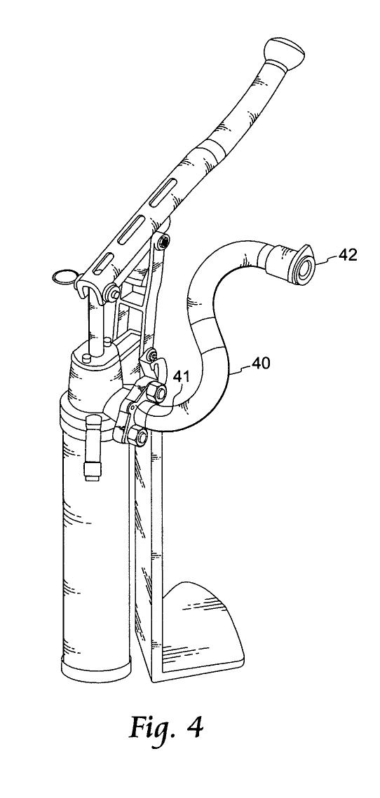

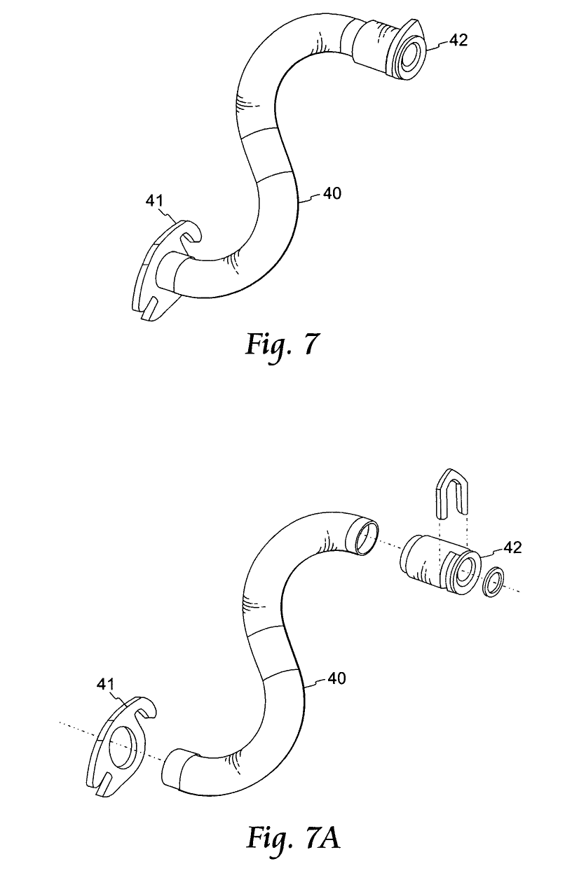

To avoid the aforesaid distortion, a high filler (40) is attached to the pump, as seen in FIG. 4. In one embodiment, shown in FIG. 7, the high filler is an S-shaped stainless steel tube with a 1 inch inside diameter, an overall length of 11½ inches, and a vertical rise of 7¾ inches between the apertures at either end of the tube. One end (41) is matched to the fitting at the output aperture (109) and the other end (42) is matched to the tool to be filled, positioned at approximately the height of the handle (104) when extended horizontally. With the high filler in place, the pump operator is able to stand more comfortably while holding the empty tool and the pump handle simultaneously during a fill operation. See FIG. 6. It has been observed that the working position is more comfortable if the high filler is mounted at a slight angle rather than vertically, as seen in FIGS. 4 and 6. Workable variants of the high filler range from an overall length of a few inches to about 20 inches.

Another improvement relates to the pump shaft. In prior art pumps, a shaft guide is provided in the top of the pump head, often a ¾ inch gland nut. This guide allows the shaft to wobble and introduces horizontal forces on the gland nut when the handle is pumped. The forces in turn cause unnecessary wear on the cylinder, piston cap and gland nut, and increase the resistance to pump motion.

The inventive design improves the pump's durability and ease of use by effectively lengthening the shaft guide elements. A longer gland nut, 1½ inches rather than the industry-standard ¾ inch gland nut, is placed in the top of the pump head. In addition, a 1½ inch gland nut is positioned in the bottom of the pump head in the flow of the mud being pumped. This combination creates a shaft guide about 4½ inches long, decreasing wobble and more efficiently directing the force from the handle vertically. The result is an easier pumping action and reduced wear on the pump cylinder, piston cup and gland nut.

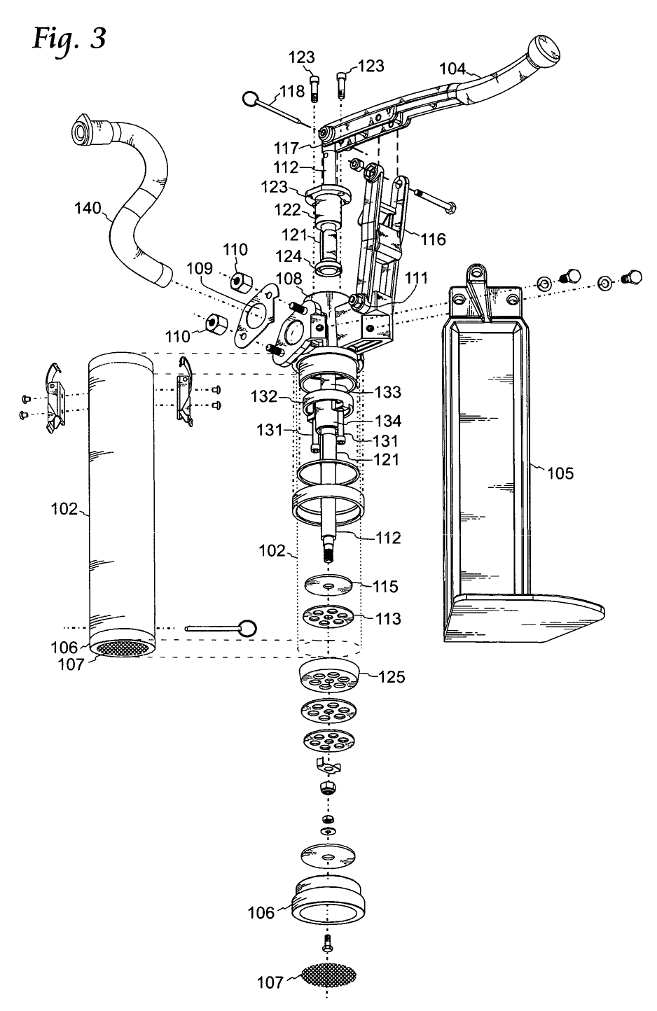

One embodiment of the invention is shown in exploded view in FIG. 3. Main body tube (102), foot valve (106) and mesh screen (107) are similar to prior pumps, as is the foot plate (105). Piston shaft (112) connects to piston cup (113) enclosing valve disc (113), making up the piston head. The top end of piston shaft (112) connects to the base (117) of handle (104) with a quick connect pin (118).

Link (116) connects handle (104) to the pump head enclosure (108) at link base (111). As previously described, link (116) is approximately 6 inches long, which is 2 inches longer than the corresponding link in prior art pumps, and link base (111) is attached to pump head housing (108) about an inch higher than in prior art pump configurations.

The upper shaft guide (123) is a gland nut attached to the head housing (108) with threaded bolts (123). Bushing liner (121) and U-cup (124) complete the upper assembly. The output aperture (109) is surrounded by a mounting bracket incorporating a pair of mounting nuts (110) that match standard tools the pump is designed to fill.

The lower shaft guide (132) is also a gland nut that secures to the head housing (108) with threaded bolts (131). A liner (133) reduces shaft wear. The high filler (140) attaches to the output aperture (109) via the mounting nuts (110).

Although the invention has been described with respect to a specific embodiment, persons of ordinary skill in the art will readily understand that the inventive concepts may be applied to a variety of configurations.

I claim:

1. A drywall mud pump comprising a main tube, a foot valve, a piston having a valve head and a shaft with a lower end and an upper end, a pump head housing having an output aperture, a lower opening to the main tube and an upper opening through which the shaft passes, and a handle attached to the shaft at an upper extremity and connected by a link to a point on the pump head housing, wherein the link is approximately six inches long and is attached at a point on the pump head housing such that the handle in a horizontal position is approximately 23 inches above the foot valve.

2. The pump of claim 1 further including an extended upper shaft guide approximately 1½ inches long attached to the pump head housing in proximity to the upper opening.

3. The pump of claim 2 further including a lower shaft guide attached to the pump head housing in proximity to the lower opening.

4. The pump of claim 2 wherein the upper shaft guide is a gland nut.

5. The pump of claim 3 wherein the upper shaft guide is a gland nut.

6. The pump of claim 3 wherein the lower shaft guide is a gland nut.

7. The pump of claim 3 wherein the upper shaft guide is a gland nut and the lower shaft guide is a gland nut.

8. The pump of claim 1 further comprising a detachable high filler configured to mount to the output aperture so that drywall mud flowing through the pump passes through the high filler and exits the pump at a point above the output aperture.

9. The pump of claim 3 further comprising a detachable high filler configured to mount to the output aperture so that drywall mud flowing through the pump passes through the high filler and exits the pump at a point above the output aperture.

10. A drywall mud pump comprising a main tube; a foot valve; a piston having a valve head and a shaft with a lower end and an upper end; a pump head housing having an output aperture, a lower opening to the main tube and an upper opening through which the shaft passes; a handle attached to the shaft at an upper extremity and connected by a link to a point on the pump head housing, wherein the link is approximately six inches long and is attached at a point on the pump head housing such that the handle in a horizontal position is approximately 23 inches above the foot valve; an extended upper shaft guide approximately 1½ inches long attached to the pump head housing in proximity to the upper opening; a lower shaft guide attached to the pump head housing in proximity to the lower opening; and a detachable high filler configured to mount to the output aperture so that drywall mud flowing through the pump passes through the high filler and exits the pump at a point above the output aperture.
